(19) United States
(12) Patent Application Publication
(10) Pub. No.: US 2015/0033628 A1
(43) Pub. Date: Feb. 5, 2015

(54) On Demand Modular Ingress/Egress Control Mechanism

(71) Applicant: Aegys, LLC, Plainfield, IL (US)

(72) Inventors: Joseph R. Barwick, Lemont, IL (US); Stephen F. Blackler, Plainfield, IL (US)

(21) Appl. No.: 14/445,268

(22) Filed: Jul. 29, 2014

Related U.S. Application Data
(60) Provisional application No. 61/860,190, filed on Jul. 30, 2013.

(51) Int. Cl. A61B 5/00; E05D 15/58; E01F 13/06; E05F 15/12; G08B 21/22; E06B 11/02; E05D 15/48; E05F 15/20

(52) U.S. Cl. CPC A61B 5/0046; E05D 15/48; E05D 15/58; E05F 15/203; E05F 15/127; G08B 21/22; E06B 11/025; E01F 13/06; E05F 2015/2061; E05F 2015/2069
USPC 49/13; 49/149; 49/124; 49/70; 49/26; 49/25

(57) ABSTRACT

A room access control system including a base attachable to a wall or door jamb adjacent a door opening to a room, an arm having a first end pivotally mounted to the base and a second end, wherein the arm is positionable in a first position wherein the arm is in a generally vertical, undeployed position with the second end of the arm positioned above a floor located adjacent the door opening, and wherein the arm is pivotable from the first, generally vertical undeployed position, to a second generally horizontal, deployed position, where the arm extends across the door opening.

### BACKGROUND

The present application relates to a modular system of controlling ingress and egress from a restricted or dangerous premises as found in hospitals, medical facilities and other settings. The disclosed embodiments provide a means to clearly communicate which premises are off limits to persons or equipment in addition to deploying a modular retractable barrier.

Many processes used in health care, industrial, and commercial fields require limited access to a room or portion of premises either at all times, or only at limited times. While one solution is to simply close or lock the door to the limited access premises, doing so isolates the limited access premises and individuals working in that area from the rest of the building and results in inaccessibility.

Within the MRI environment there is a superconducting magnet encapsulated in its own specific room. This room must be accessed through an RF shielded door. This door is kept open at certain times for a variety of clinical reasons including patient flow, medical staff egress, emergency situations and simple communication outside the room. An industry acknowledged exposure to MRI technologists and the patients they image on a daily basis is bodily injury or death resulting from a projectile accident occurring. A projectile accident is defined as an occurrence where an object containing ferromagnetic material is pulled into the superconducting magnet at a high rate of speed.

As shown in photograph of FIG. 1, labeled as Prior Art, MRI suites are generally protected with passive signage, and in some instances, illuminated signs indicating the presence of a magnetic field. However, as shown in FIG. 1, the standard signage may use a green illuminated sign 1 displaying various warnings directly over the doorway 2 of door 3. Most visitors or even facility staff members do not understand the danger caused by entering the room where the high-intensity magnetic field is located. Other symbols on doors may be used, however, they do not convey the danger and are not sufficiently active to guarantee attention of the viewer.

A device which controls access to the room, protecting persons and equipment in that room and unequivocally communicates the danger found within the room without entirely isolating the room overcomes the aforementioned problems.

A need exists in the art for a means to control physical access to a premises without completely isolating the premises. The method and system should rely on active information signage that clearly communicates to any person that the premises of a given room is off limits and physically restricts its access with a retractable physical barrier.

### SUMMARY

In one aspect, a room access control system is provided that includes a base attachable to a wall or door jamb adjacent a door opening to a room, an extending arm having a first end pivotally mounted to the base and a second end, wherein the extending arm is positionable in a first position wherein the arm is in a generally vertical, undeployed position with the second end of the arm positioned above a floor located adjacent the door opening, and wherein the extending arm is pivotable from the first, generally vertical undeployed position, to a second generally horizontal, deployed position, where the arm extends across the door opening.

### BRIEF DESCRIPTION OF DRAWING

The invention together with the above and other objects and advantages will be best understood from the following detailed description of the preferred embodiment of the invention shown in the accompanying drawings, wherein:

FIG. 1 illustrates a prior art notification system for limited access premises;

FIG. 2 illustrates one embodiment of an access control system 10, in accordance with features of an example embodiment;

FIG. 3 is a front view of the base 12 of the access control system 10 of FIG. 2, in accordance with features of an example embodiment;

FIG. 4A is a cross-sectional view of the base 12 of the access control system 10 shown in FIGS. 2 and 3 taken along line 4A-4A in FIG. 3;

FIG. 4B is an exploded view of the base 12 shown in FIGS. 2 and 3;

FIG. 4C is a bottom view of base 12 shown in FIGS. 2 and 3;

FIG. 5A is a perspective view of a base assembly 70 of the base 12 shown in FIGS. 2-4C;

FIG. 5B is a perspective view of a hinge 46 shown in FIG. 3, in accordance with features of an example embodiment;

FIG. 6A is a perspective view of a base segment 90 that may be used with arm 22 shown in FIG. 2, in accordance with features of an example embodiment;

FIG. 6B is a perspective view of telescoping components that may be used with arm 22 shown in FIG. 2, in accordance with features of an example embodiment;

FIG. 6C is a perspective view of a linear actuator 100 that may be used with a telescoping arm, in accordance with features of an example embodiment;

FIG. 6D is a perspective view of a foam tip 110 that may be used as a component of the telescoping arm, in accordance with features of an example embodiment;

FIG. 7 is a perspective view of the linear drive 52 shown in FIGS. 3 and 4A, according to an example embodiment;

FIG. 8 is a perspective view of mounting plate 130 that may be used as wall plate 16 shown in FIG. 2, in accordance with features of an example embodiment; and

FIG. 9 is a perspective view of support bracket 140 that may be used as a support for base 12 shown in FIG. 2; in accordance with features of an example embodiment.

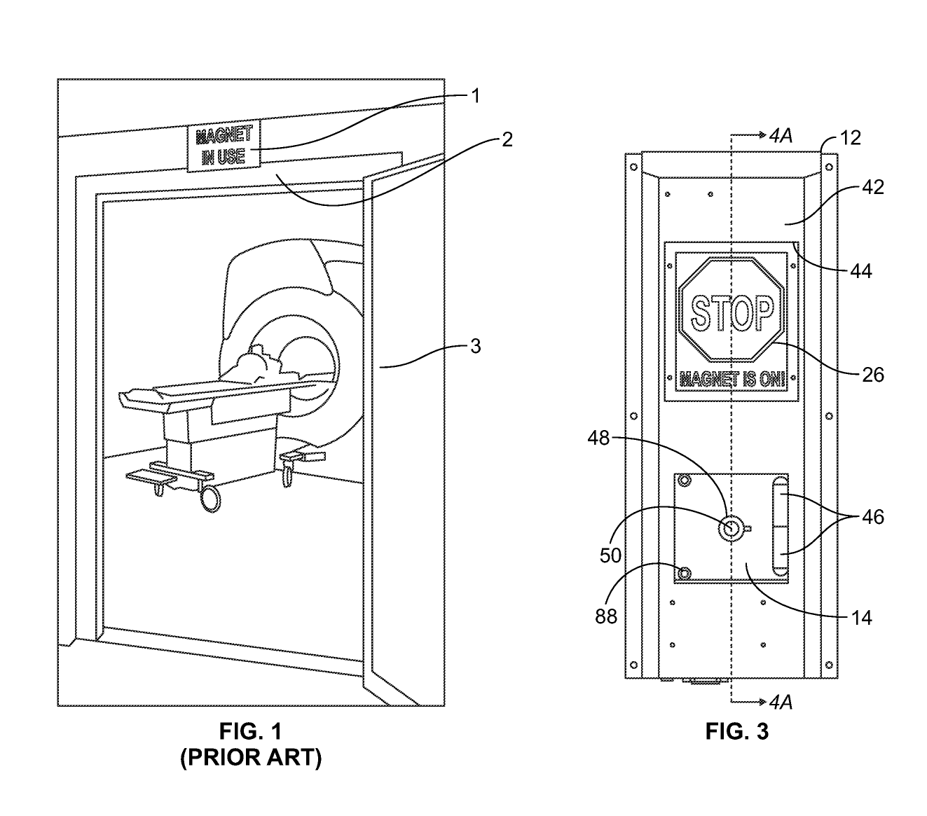

### DETAILED DESCRIPTION

The foregoing summary, as well as the following detailed description of certain embodiments of the present invention, will be better understood when read in conjunction with the appended drawings.

As used herein, an element or step recited in the singular and preceded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

An embodiment of the system controls access to a room by being mounted on the side of a door or a door jamb or a door opening. The system may include a telescoping arm, which pivots around a fulcrum point. In an un-deployed position, the arm is oriented vertically, i.e. in a parallel with vertically disposed portions of a door jamb, the arm defining a first length. Upon deployment, the arm first pivots upwardly (or downwardly) to a horizontal position such that the arm is now orthogonal to the vertically disposed portions of the door jamb. Upon attaining this position, the arm, may thereafter be extended automatically to a second length to cover the width of the door. (In an embodiment of the invention, extension to the second length occurs while the arm is positioning from a vertical to a horizontal position).

During and following deployment warning indicia are illuminated on the system. Indicia can be illuminated at all aspects of arm deployment, for example, just before deployment of the vertically disposed first length arm to its horizontal position, or at the beginning of deployment, or during extension to the second length, or only when the system attains final second length deployment status.

While described in the context of an MRI suite, the instant device can be deployed in conjunction with any process where access control is desired while maintaining an open or partially opened door.

One embodiment provides for a deployment completion audible tone or melody upon deployment, upon activation or during extension of the arm the second length or attainment of the final second length deployment status.

One embodiment provides for a modular room access control system, designed to notify third parties of a danger located within a premises. While the depicted embodiment is focused on limited access to MRI suites, the device is capable of protecting any limited access rooms or buildings. For example, one embodiment, not shown, is extended over a door while the premises are being flooded with ultra-violet light for disinfection purposes. Another embodiment, not shown, is extended over the entrance to a ‘clean room’ environment. In such an instance, the system is used in conjunction with a sealed door.

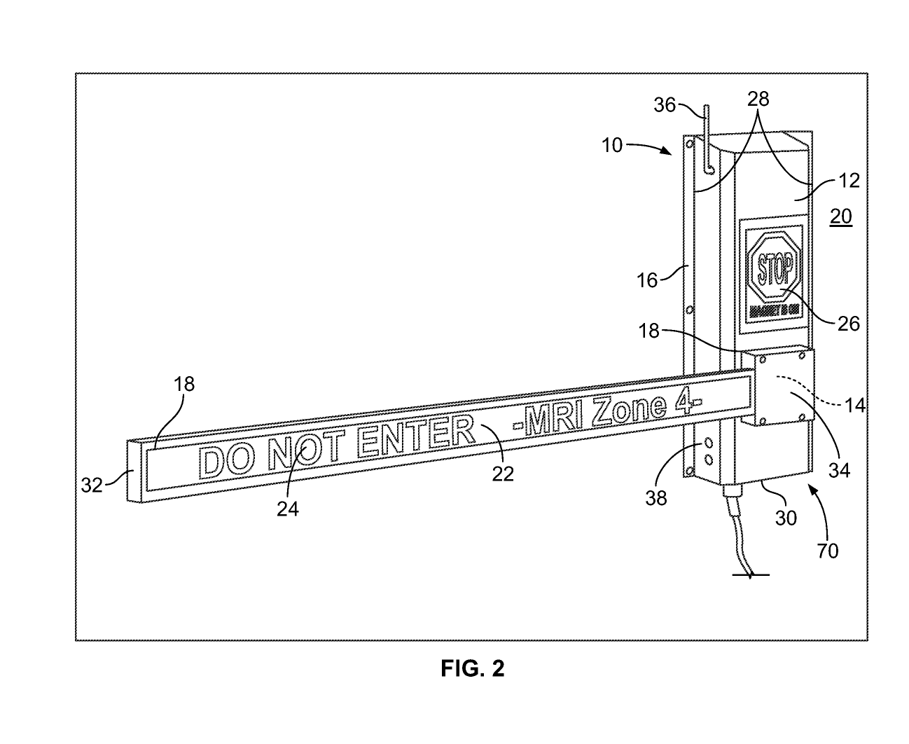

As shown in FIG. 2, one embodiment of the access control system, designated as numeral 10 comprises a base 12 in rotatable communication with an arm receptacle 14. The base is mounted to a wall plate 16, and the wall plate 16 is in turn attached to a wall 20. The base 12 includes a base assembly or housing 70 that substantially encapsulates internal componentry of the access control system 10 and electrically isolates the componentry from regions exterior of the base assembly or housing 70. Portions of the housing 70 can provide a means for pulling heat away from the componentry so as to act as a heat sink.

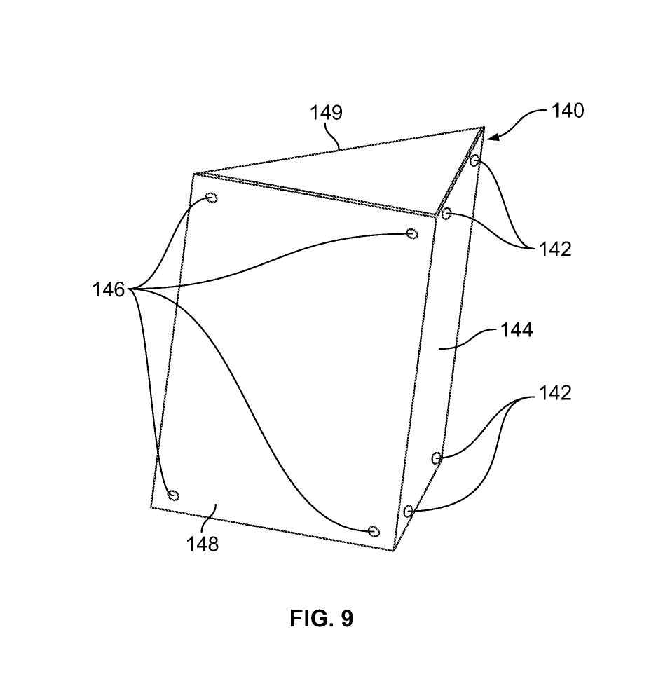

The base 12 can be mounted on either an in-swing or out-swing door opening, specifically on the hinge-side or non-hinge side of an in-swing door or the non-hinge side of an out-swing door opening. Furthermore, some MRI/Medical doorways have a perpendicular wall on one side of the doorway or a corridor leading to a door opening. For addressing this situation an L shaped bracket, as shown in FIG. 9, allows the system to be mounted securely and function in the same way as it would if mounted on the side of the door.

An arm assembly 18 may be removably attached to the base 12 at the receptacle point 14. For example, the arm assembly 18 may be slidably received by the base 12, or else received in a snap fit configuration by the base 12, or magnetically coupled to the base 12.

In the embodiment shown in FIG. 2, the arm assembly 18 comprises an arm 22 that may be made from a light weight material selected from the group consisting of acrylic, aluminum, wood, carbon fiber, fiberglass and combinations thereof. The arm 22 displays warning indicia 24. Optionally, outwardly facing surfaces of the base 12 display warning indicia 26. Additionally, perforations or holes may be formed on the back side of arm 22 to illuminate visibility of the arm to those persons approaching the deployed arm from the interior of the space being restricted.

The arm assembly 18 as shown in the embodiment of FIG. 2, comprises an arm 22 with a first end terminating in an end cap 32 and a second end in pivotal communication with a region of the outwardly facing surface of the base 12 defining a pivot point 34. The pivot point 34 may define a nut-bolt configuration or a snap fit configuration the latter of which to provide a reversible attachment means of the arm 22 to the base 12. The removable attachment facilitates the disengagement of the arm 22 from the base 12 in the event of an emergency or inadvertent collision, and is shown in detail below. Further, the removable attachment is truly modular allowing for the repair or upgrade of the arm 22.

The arm 22 pivots around the fulcrum attachment point 34 from an un-deployed position which is parallel to the sides 28 of the base 12 (and parallel to the vertically disposed portions of the door jamb) to the deployed position wherein the arm 22 forms an angle α to the sides of the base 12. While FIG. 2 shows the arm as substantially perpendicular to the longitudinal sides 28 of the base 12, a myriad of angles are suitable, ranging from about 45 degrees to about 135 degrees. An embodiment of the deployed configuration is shown in in FIG. 2.

During the deployed configuration in FIG. 2, the arm indicia 24 and the base indicia 26 are illuminated. In one embodiment, the arm indicia 24 and the base indicia 26 flash, remain constant, or otherwise illuminate following deployment. In further embodiments multiple colors are utilized to correspond with differing stages of deployment.

A noted supra, in another embodiment, the sound generating component of the base 12 is engaged during the deployment process when the arm 22 is switching from the un-deployed vertical configuration to the deployed configuration shown in FIG. 2.

In one embodiment, the base 12 further comprises a radio frequency antenna 36 for receiving wireless signals from a remote transmitter (or vice versa where the base incorporates a transmitter to communicate with a receiver). The arm 22 may be deployed or un-deployed in response to receipt of a wireless communication signal by control circuitry found within the base 12 as captured by the antenna 36. In one embodiment, the antenna 36 receives unencrypted signals over industry-standard frequencies such as those not subject to national regulation, i.e. 900 Mhz and 2.4 Ghz and 5 Ghz. Optionally, the antenna 36 receives encrypted signals from the remote.

In one embodiment, a side 28 of the base 12 includes manual actuation buttons 38 which can be used to deploy or un-deploy the arm 22. The buttons 38 may also be used to select an encryption key for the wireless signal. In this embodiment, when both keys are pressed, the control circuitry within the base 12 selects a random encryption key and broadcasts it using the antenna 36. The encryption key is received by the remote. Upon acknowledgement of receipt of the encryption key by the remote, the control circuit ceases sending out of the encrypted key.

In one embodiment, the encryption keys are set by a series of dip switches in the remote and on the base. In order to function, banks of corresponding dip switches must be set to the same value.

In one embodiment, the base 12 is powered by a standard household current, 110-130V, with a power plug extending from an exterior surface of the base, such as the bottom surface 30 of the base 12. Inasmuch as during operation, the device preferably does not exceed 2.75 amps of current, the system is amenable to being powered by a backup power source, such as an off-the-shelf uninterruptible power supply or a low current generator. In another embodiment, the base 12 is powered by a direct current battery, such as standard batteries 12V batteries used with cordless tools. This DC configuration is particularly applicable when the system is used as a completely modular unit, so as to be wheeled from passageway to passageway, as needed. In this configuration, the system may be placed on a cart along with its power supply. The power supply can be reversibly attached to the base of the system for cosmetic purposes, or else in electric communication with the system via standard insulated conductors.

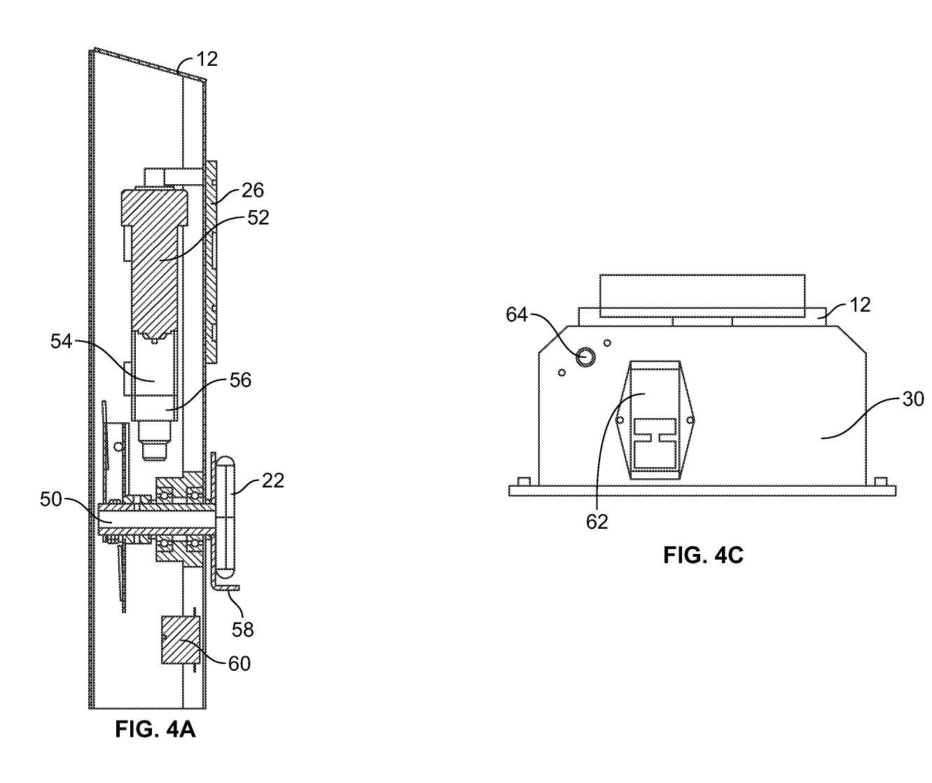

Turning to FIG. 3, a front view of the base 12 of the system 10 is depicted. The base 12 includes a front plate 42 which secures the interior components of the base 12 discussed herein, so as to provide a means for electrically insulating the components from passersby. The front plate 42 includes a mounting point 44 for the base warning indicia 26. In the embodiment shown in FIG. 3, the mounting point facilitates the installation of any number of removable warning indicia 26. The warning indicia 26 can be added or removed depending on the desired cautionary message to be displayed thereon. In the embodiment shown in FIG. 3, the warning indicia 26 cautions against the danger of the magnetic field, but could include other messages. In one embodiment, the indicia mounting point 44 includes removable attachment means, such that the indicia 26 can replaced in the field, as the base 12 is moved from one application to another. For example, as shown in FIG. 3, the warning indicia 26 can be bolted on using screws or other threaded members. The removable attachment of warning indicia 26 to the mounting point 44 also allows for the replacement of the indicia 26 in the event that the indicia ceases to illuminate, or in the event that brighter illumination is required or becomes feasible. Also, warning indicia may be modular, for example a low-powered LED with its own power source can be removably attached to the housing such as via magnets, hook and pile connectors (e.g. Velcro) or with a simple elastic band adapted to encircle the housing unit.

In another embodiment, the attachment means are designed to be operable only in one direction, such as with anti-theft fasteners so as to allow fastening to the faceplate of the housing and prevent the unauthorized removal of the warning indicia 26 or other defacement.

The front plate 42 further includes an arm receptacle 14. The receptacle 14 includes a pair of weld-on hinges 46, discussed in detail below. The arm receptacle includes a keyed aperture 48 containing the arm actuator pin 50. The aperture is keyed to ensure that the arm is installed in the correct orientation, thereby deploying from a vertical position to a horizontal. Alternatively, and as discussed supra, the receptacle facilitates magnetic interaction with a ferrous containing portion of the arm.

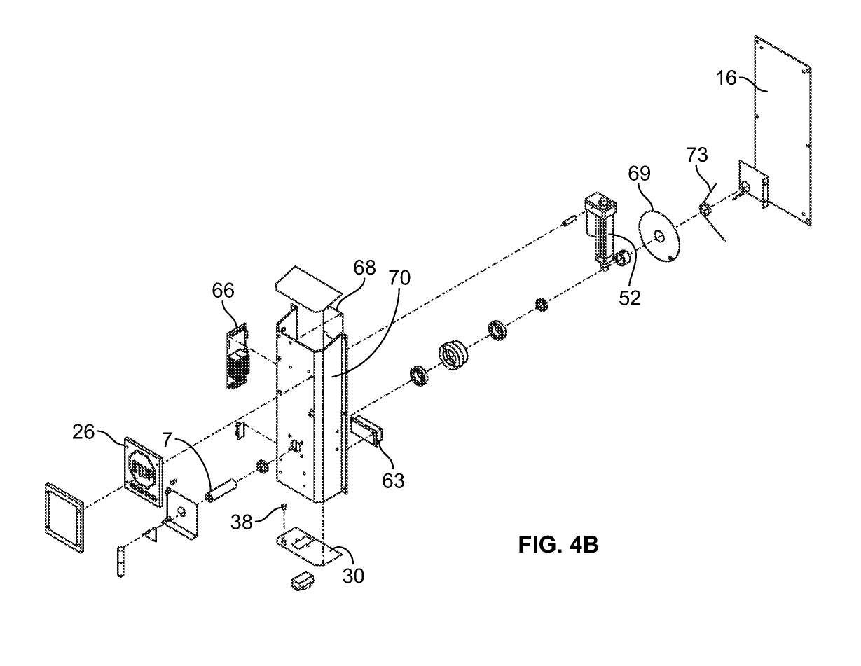

Turning to FIG. 4A, depicted therein is a schematic view of the interior of the base 12 taken along lines 4A-4A of FIG. 3. Installed within the base 12 is a linear drive 52. The linear drive 52 comprises a cylindrical body 54 and drive element 56. The drive element 56 is affixed to an off center edge of a round plate (69 in FIG. 4B) which upon extension of the drive element 56, rotates the plate. The arm actuator pin 50 is affixed to the center of the round plate (69 in FIG. 4B) and the rotation of the plate 69 rotates the actuator pin 50 which in turn deploys the arm 22. The drive element 52 is further connected to the cautionary indicia 26 and therefore the indicia 26 are illuminated when the drive element 56 is extending.

In one embodiment, there are mechanical limit switches which are set on the linear drive that communicates the relative position of the arm from disengagement, active deployment, to engagement and back again. A logic controller runs the program to activate the cautionary indicia to correspond with the position or activity of the arm.

Optionally, as a counter weight to the arm 22 and to increase rigidity of the arm 22, a support plate 58 is installed around the base of the arm 22.

Power and control circuitry is located within the base 12 in a replaceable module 60.

Turning to FIG. 4B, the power supply 63 provides electrical power to the control board 66 which in turn runs a stored programmed set of instructions. The instructions are executed in response to input from the button 38 or the RF receiver 68. Upon activation from either element, the linear drive 52 extends which rotates the round plate 69 which in turn rotates the attached arm. The plate 69 is under spring loaded tension from spring 73 which controls the velocity of the rotation and position. Limit switches on the linear drive 52 provide position data to the control board 66 to activate the display flash for warning indicia 26 or illumination color changes on the arm. In the case of the extendable arm, the limit switches in the linear drive 52, communicate when the rotation has completed to horizontal upon which case the telescoping linear actuator in the arm is activated to extend the arm. The system is modular and the base can be used with either a non-telescoping lit arm or a telescoping, non-illuminating arm. Additionally, in alternative embodiments, upgraded arms are designed to operate with the master base. A connector detector of voltage allows for the base to recognize which arm has been attached and to activate the appropriate operational programs stored on the control board. In another embodiment, each arm includes an encrypted identifier to signify which arm has been installed on the base.

In one embodiment, the system includes an ultrasonic sensor that will monitor for the presence of someone standing in the arm deployment path that will prevent operation upon detection of a person or object. Additionally, a voltage monitoring chip monitors the operation of both the linear drive and telescoping linear actuator for spikes in current associated with resistance (if the arm were to come in contact with an object) and if pre-set thresholds are reached, the system will reverse the current operation until either a default engagement or disengagement state is achieved.

Turning to FIG. 4C, the bottom plate 30 of the base 12 is depicted therein. A power socket 62 is located on the bottom plate 30. The power socket 62 accepts using a friction fit of a standard power cord, in one embodiment wherein the socket 62 is a C13 receptacle accepting IEC 60320 compliant power cords. The bottom plate 30 further includes an LED indicator 64 to show that control circuitry is receiving power and is operating correctly.

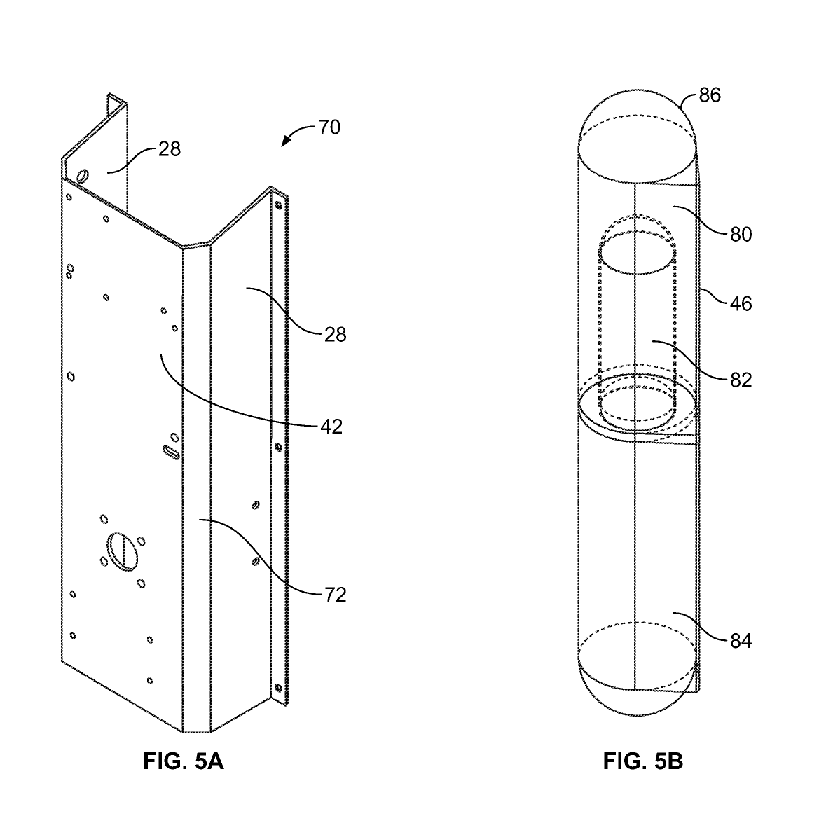

Turning to FIG. 5A, shown there is an embodiment of a base assembly 70 for the base 12. The base assembly 70 defines the sides 28 and front plate 42 as a single piece to facilitate ease of manufacturing. Corners 72 formed at the intersection of the sides 28 and the front plate 42 are tapered to eliminate sharp edges where a user may be injured. In another embodiment, not shown, the corners formed by the joining of the base assembly 70 and the top surfaces are likewise tapered.

Turning to FIG. 5B, depicted therein is a weld-on hinge 46. In the embodiment shown in FIGS. 3 and 4A, the support plate 58 includes two weld-on hinges 46. Each weld-on hinge 46 comprises a first section 80 and a second section 84. A smaller internal body 82 is located within the first section 80. Each of the first section 80, the second section 84 and the internal body 82 are capped with a half-spherical body 86. The weld-on hinges 46 facilitate the separation of the arm 22 from the base plate 58 in the event of an emergency.

Essentially, the bottom 84 of the hinge 46 will be mounted to a plate that is attached to the base 12, with the top 80 of the hinge 46 being attached to a plate that is on the back of whatever arm is being utilized. The hinge allows for the arm to swing parallel to the ground and into operational position at which point vinyl or plastic screws or bolts may be used to “sandwich” the plates together to hold the arm in position during operation. These screws or bolts would flex or destroy when pressure is placed on the arm rotating it parallel to the ground—allowing for emergency, manual override, upgrade or repair of the arm. The location of these hinges is designated on FIG. 3 as location 88.

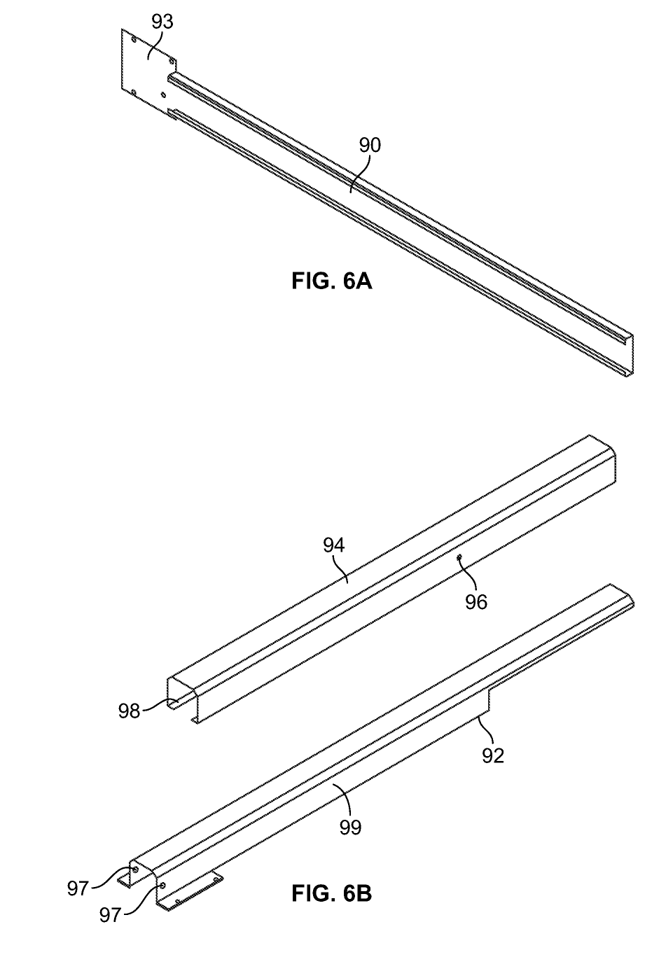

Turning now the FIG. 6A, the arm base segment 90 of the arm 22 is shown therein. The base segment 90 is used in a non-telescoping arm embodiment, with a vinyl illuminated cover which may be used thereon. The cover in one embodiment is an etched acrylate. The base segment 90 includes a fulcrum point 93, which attaches the base segment 90 to the device base 12 as shown in other figures.

As shown in FIG. 6B, in another embodiment, the arm features a telescoping action. The arm extension 94 of one embodiment of the telescoping arm is depicted in FIG. 6B. The arm extension 94 includes an aperture 96 designed to receive the extending mechanism described herein. The arm extension 94 comprises opposing rails 98 designed to be removably and slidably received by the rails 92 of the base segment 99. The attachment point 96 would attach to the end of the actuator 100 and would extend upon activation. The actuator 100 is also covered by the base segment 99, which covers the actuator at all times and is more visible upon deployment and resides within the interior of extension 94 when not extended.

Alternatively, the arm 22 can define a plunger-cylinder configuration, whereby the arm extends when the plunger actuates and is pushed outwardly.

Turning now to FIG. 6C, the linear actuator 100 is depicted therein. The linear actuator is attached to the fulcrum point of the base segment 90 and the aperture 96 of the extension 94. Upon activation of the linear actuator 100 the extension 94 moves along the rails 92 of the base segment 90. The end 106 of actuator 100 is attached at point 96 in FIG. 6B, while end 107 is attached to point 97 of FIG. 6B.

FIG. 6D depicts a foam tip 110 of the assembled arm. In one embodiment, the extension 94 continues to extend outwardly away from the base 90 fulcrum point until the foam tip 110 touches an opposing surface, such as a door or wall frame. The foam tip 110 does not include a sensor. Instead, the linear actuator 100 is sensitive to the resistance from the foam tip 110 and will stop extending the extension 94 upon encountering resistance on the foam tip 110. The extension therefore does not include any sensor or switch, instead it is capped with a simple foam tip 110 thereby avoiding complicated circuitry within the telescoping arm.

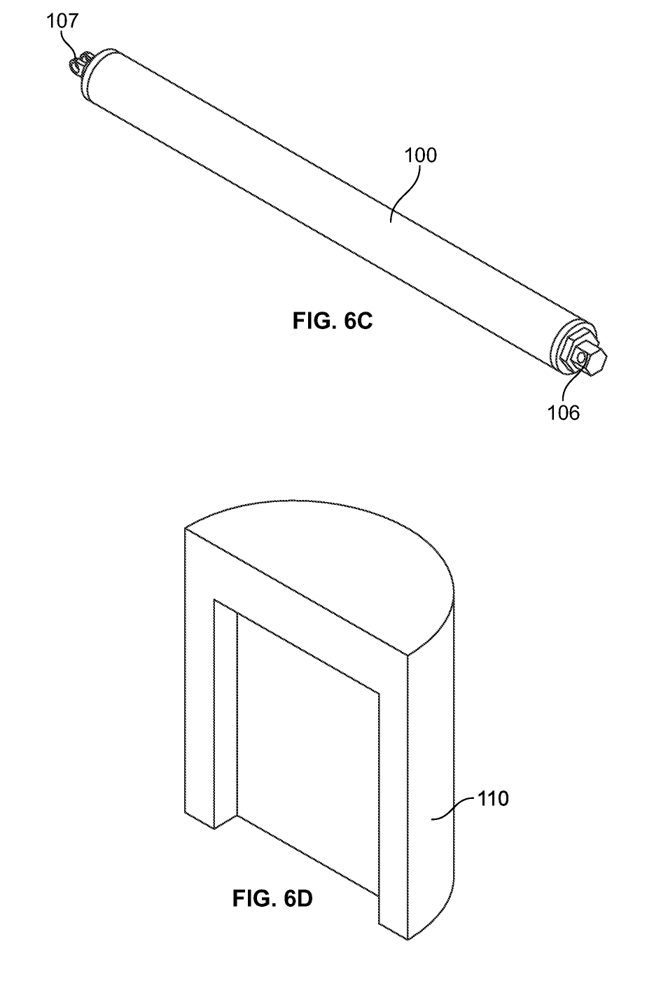

FIG. 7 depicts an additional view of the linear drive 52. The linear drive 52 converts the rotational movement of the motor into a linear movement which is used to extend the telescoping arm. The linear drive 52 includes a control enclosure 120, the motor 122, and the linear drive element 124. The linear drive is attached to the base 12 at the linear drive pedestal 126. In one embodiment, the linear drive 52 is a drop-in replaceable component with a mean time between failures of 20,000 cycles. In one embodiment, a linear drive from Duff-Norton Corporation, Model: TMD01-1906-D is used as the driving module.

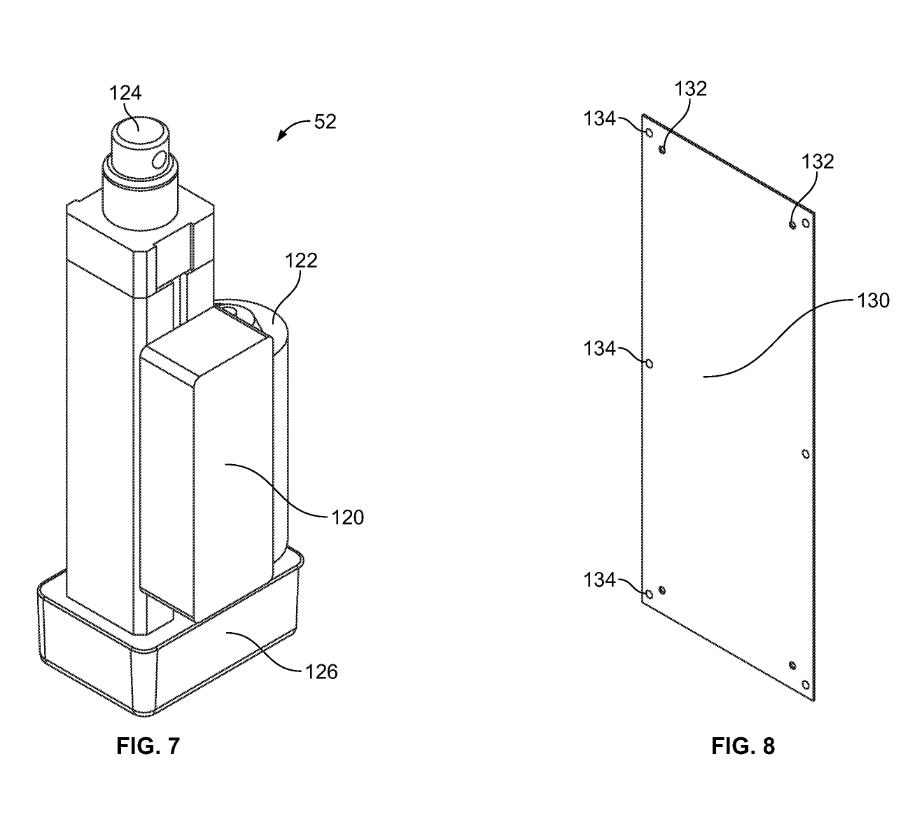

FIG. 8 depicts a wall mounting plate pursuant to an example embodiment. As shown in FIG. 8, the mounting plate 130 may be used as wall plate 16 shown in FIG. 2, and may be attached directly to the wall, using apertures 134 adapted to receive any standard dry wall anchor, screw etc. The base assembly 12 in turn is connected to the mounting plate 130 through apertures 132, which in an example embodiment may be threaded posts. The apertures 134 are spaced to correspond to locations of reinforcement studs within a standard commercial wall. In another embodiment, the apertures 134 are shaped to allow for mounting of different threaded members, such as ones optimized for anchoring to brick, drywall, metal, and wood studs. It is to be appreciated that inasmuch as the MRI systems must be electrically isolated from electromagnetic interference, MRI enclosures are typically encased in ferrous materials. As such, a magnetic mounting system for the system directly to the enclosure surface is a suitable alternative, particularly in instances where one system is to be used in different locations on the fly.

FIG. 9 depicts an alternative embodiment of support bracket 140 using a welded “L” support bracket shape that may be used to support base 12. The alternative support bracket 140 uses affixment points 142 on a first face 144. These affixment points 142 are for wall mounting. Further, a second face 148 of the bracket 140 includes threaded studs 146 for mounting on the base of the product. Finally, the bracket 140 includes a third reinforced angle section 149 for support.

The embodiments disclosed herein advantageously provide an ingress and egress control method that overcomes many of the disadvantages of the prior art. The disclosed embodiments may provide caution indicia for a premises that is impossible to overlook, ignore, or unintentionally bypass. In some embodiments, the use of a telescoping arm with warning indicia is employed. An advantage of the disclosed embodiments is that any third party observer will understand the danger involved in entering the protected premises and will not accidentally wander into same. A further advantage of the disclosed embodiments is the providing of an arm that may extend over the entire width an opening without taking up excess space while the arm is in an un-deployed configuration. Further, the system may use a telescoping arm which pivots around a fulcrum point to extend over the entirety of the door. An advantage of a telescoping arm is that the arm prior to pivoting and extending does not require an excess amount of vertical clearance.

The disclosed embodiments provide an access control mechanism which does not impede communication, and may include a telescoping arm that extends over an open or partially open door. In addition, the disclosed embodiments allow for persons located in the secured premises to remain in visual, aural and fluid communication with those outside.

The present embodiments may also include the addition of a manual override switch which can be used in emergency situations or if the remote control functionality is somehow impeded. A safety feature may be provided of a side mounted ultra-sonic sensor that ensures no person or object is in the threshold of the door when operation of the modular arm is initiated. Further, the use of a voltage monitoring chip may be used to measure resistance on the arm during deployment to ensure that collisions are mitigated.

The disclosed embodiment may provide an access control device which can be removed in an emergency situation. For example, a break-away joint between a telescoping arm and its base may be used. Furthermore, the arm may be reversibly removed from the base to access the room in an emergency, without permanent damage to the telescoping arm. In addition, the disclosed embodiments may allow for simple retrofitting of existing premises to add access control systems. The access control system may be modular such that it can be installed on either side of a door, on any perpendicular wall, embedded in the construction of a wall or deployed on a mobile cart in an example embodiment. An advantage of the disclosed embodiments is that the access control system can be installed alone, or in tandem with another similar module. Another advantage is that the access control system can be installed on the premises that were originally designed without such deployments (and the associated power routing requirements therewith) in mind.

The disclosed embodiments provide for a variety of triggers that may be used for activation. For example, activation by be triggered by a smart phone AP trigger, an RFID trigger, a Bluetooth RFID trigger, a proximity trigger, a Ferromagnetic Detection trigger, a broken infrared beam trigger, or a camera trigger, as examples. Furthermore, the disclosed embodiments may include internet connectivity for monitoring, remote programming, among other functionality, and may include date exporting functionality.

In addition, the disclosed embodiment may include a time measuring trigger for activation, and may include integration within a door, door jamb or integration with door movements as a trigger for activation. In addition, the disclosed embodiments may include an extendable arm link into a locking mechanism upon deployment for secure access control, and may also provide an audible signal when extended.

Further, the disclosed embodiments may be embedded within a wall or wall cavity for a reduced profile. The disclosed embodiments may also include rear indicators on the extended arm for visibility of the arm from within the space being restricted. A battery backup for power outages may also be provided.

The disclosed embodiments may provide a modular room access control system that may include a telescoping or fixed arm wherein said arm is adapted to pivot about a fulcrum point from a vertical position to a horizontal position and in the case of a telescoping arm may then to extend from a first point to a second point. The arm may also include a means for reversibly detaching the arm from the fulcrum point.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

The embodiment of the invention in which an exclusive property or privilege is claimed is defined as follows:

1. A room access control system comprising:
a base attachable to a wall or door jamb adjacent a door opening to a room;
an arm having a first end pivotally mounted to the base and having a second end;
wherein the arm is positionable in a first position wherein the arm is in a generally vertical, undeployed position with the second end of the arm positioned above a floor located adjacent the door opening; and
wherein the arm is pivotable from the first, generally vertical undeployed position, to a second generally horizontal, deployed position, where the arm extends across the door opening.

2. The room access control system of claim 1 wherein the arm comprises a telescoping arm wherein an arm extension is extendable from the second end of the arm to increase the overall length of the arm.

3. The room access control system of claim 2, wherein the arm is pivotable from the second, generally horizontal deployed position back to the first, generally vertical undeployed position, and wherein the arm extension is retractable.

4. The room access control system of claim 1 further comprising illuminated warning indicia positioned on the arm cautioning against entry through the door opening when the arm is in the second, generally horizontal deployed positioned.

5. The room access control system of claim 2 wherein the extension of the arm extension ceases upon contact with an opposing surface.

6. The room access control system as recited in claim 1, wherein movement of arm stops upon contact with an object, and results in movement of the arm back to the first, generally vertical undeployed position.

7. The room access control system as recited in claim 1 wherein the arm extension of the arm includes illuminated warning indicia.

8. The room access control system as recited in claim 1 wherein said base is mounted on a mobile cart.

9. The room access control system as recited in claim 1 wherein a footprint of the room access control system when mounted on the wall is less than 8½ inches by 21 inches.

10. The room access control system as recited in claim 1 wherein said system utilizes an audible tone or melody to correspond with activation, deployment or extension activity.

11. The room access control system as recited in claim 6 wherein a voltage monitor is utilized to measure resistance as a safety control during all motions of the arm and to stop movement of the extending arm upon contact with the object.

12. The room access control system of claim 1, wherein the first end of the arm is removably mountable within an arm receptacle mounted for rotation with respect to the base to allow for a replacement arm to be easily mounted within the arm receptacle.

13. The room access control system of claim 1, wherein illuminated indicia are positioned on a front surface of the base.

14. The room access control system of claim 1, wherein the arm is rotatable upon receipt of wireless signal received from a remote transmitter located within the room.

15. The room access control system of claim 1, wherein the arm is hingedly mounted to an arm support plate.

16. The room access control system of claim 15, wherein the arm is releasably secured to the arm support plate.

17. The room access control system of claim 16, wherein the arm is releasably secured to the arm support plate using a magnetic coupling, and wherein the arm may be separated from the arm support plate by pushing the arm in a direction away from the support plate to break the magnetic coupling.

18. The room access control system of claim 17, wherein after the magnetic coupling is broken, the arm may be completely separated from the arm support plate by the exertion of an upward force on a bottom of the arm.

19. The room access control system of claim 1, further including means for rotating the arm with respect to the base.

20. The room access control system of claim 1, wherein illuminated indicators are positioned on a side of the arm facing into the room to notify persons within the room that the arm is positioned in the second, generally horizontal deployed position.

21. The room access control system of claim 1, wherein movement of the arm from the first, generally vertical undeployed position, to the second, generally horizontal deployed position may be activated by using one or more triggers selected from the group of a smart phone AP trigger, an RFID trigger, a Bluetooth RFID trigger, a proximity trigger, a Ferromagnetic Detection trigger, a broken infrared beam trigger, and a camera trigger.

22. The room access control system of claim 1, further including an ultrasonic sensor as a safety feature to prevent movement of the arm when an object is sensed in the door opening.

23. The room access control system of claim 4, wherein an MRI machine is positioned within the room, and the illuminated warning indicia on the extending arm provides a warning that the MRI machine is in use.
